(19) United States
(12) Patent Application Publication
(10) Pub. No.: US 2014/0330183 A1
(43) Pub. Date: Nov. 6, 2014

(54) Orthopedic Cast and Method to Manufacture Same

(71) Applicant: Mandad KAZEMTABRIZI, Toronto (CA)

(72) Inventor: Mandad KAZEMTABRIZI, Toronto (CA)

(21) Appl. No.: 13/886,150

(22) Filed: May 2, 2013

(51) Int. Cl. A61F 5/058

(52) U.S. Cl. CPC A61F 5/05866
USPC 602/12

(57) ABSTRACT

Provided herein is an orthopedic cast and a system and method for manufacture of same. The orthopedic cast comprises a substantially rigid inside wrist portion having a palm support adapted to engage with a palm of a wearer and an inside arm guard adapted to engage with the inside of a forearm of a wearer. The cast further comprises a substantially rigid outside wrist portion having a back hand guard for engaging the back of the wearer's hand and an outside arm guard for engaging the outside of the forearm of the wearer. The inside wrist portion and outside wrist portion are adapted to be secured to the wrist of a wearer to immobilize the wearer's wrist.

### TECHNICAL FIELD

The following relates generally to an orthopedic cast.

### BACKGROUND

Orthopedic casts and splints are used to immobilize a body member having a broken bone, torn ligament, or other injury. For example, a bone fracture may heal properly if movement of the fractured bone is significantly restricted. Orthopedic casts, often referred to simply as casts, immobilize fractures and allow bones to heal in place. The healing process for a broken bone can take between 4 to 24 weeks, and the fracture is immobilized to varying degrees throughout this process.

A typical cast consists of a rigid structure that envelopes the injured body member in the area around the injury to prevent movement. Casts often completely envelop the injured body member and are typically formed of plaster or a fibre-reinforced composite. By way of example, a cast for a wrist fracture may envelop the wrist, forearm, and portions of the hand to ensure that movement of the wrist is significantly restricted or inhibited.

However, casts typically do not enable respiration of the skin and, as such, may cause infection, skin irritation, lack of hygiene, and a generally unpleasant experience for the wearer. In recent years, casts have been applied over breathable liners to mitigate these effects, however, breathable liners are of limited effectiveness. Plaster casts are also heavy, cumbersome, damaged when in contact with water, and immobilize more of the wearer's body than is necessary to immobilize the fracture. Plaster casts are also not aesthetically pleasing.

Furthermore, casts are applied by medical professionals at a medical facility and cannot be removed safely without specialized tools. As such, a wearer of a cast returns to the medical facility to have the cast removed and to periodically examine the injury to determine whether the fracture is healing effectively. If the injury requires additional time to heal, a new cast is applied to the wearer. The healing process, in some examples, requires multiple removals and re-applications of the cast. Complications may also arise due to effects caused by the cast including muscle atrophy and infection which may necessitate temporary removal of the cast. Removal and re-application of plaster casts is time consuming, inconvenient, and expensive.

Specifically, existing casts are applied, and removed using sophisticated equipment, often by trained professionals.

As such, existing casts cause significant discomfort, inconvenience, and potential medical issues, leading to an unpleasant experience for the wearer.

### OBJECT OF THE INVENTION

It is therefore an object of the present invention to obviate or mitigate at least one of the above disadvantages.

### SUMMARY OF THE INVENTION

In a first aspect there is provided an orthopedic cast having a substantially rigid inside wrist portion having a palm support adapted to engage with a palm of a wearer and an inside arm guard adapted to engage with the inside of a forearm of a wearer. The cast further comprises a substantially rigid outside wrist portion having a back hand guard for engaging the back of the wearer's hand and an outside arm guard for engaging the outside of the forearm of the wearer. The inside wrist portion and outside wrist portion are adapted to be secured to the wrist of a wearer to immobilize the wearer's wrist.

In another aspect, there is provided a method of selecting an orthopedic cast comprising obtaining a scan data file of a body member, assigning immobilization points to the scan data file of the body member, generating a spatial mapping of contact areas, each contact area being located to engage with one or more of the assigned immobilization points and selecting, from a cast template database, a cast template having contact areas that most closely correspond to the mapping.

In yet another aspect there is provided a system for selecting an orthopedic cast comprising a scanner operable to obtain a scan data file of a body member, and a cast model generator linked to the scanner. The cast model generator comprises an immobilization point locator module operable to assign immobilization points to the scan data file of the body member, a contact area generation module operable to generate a spatial mapping of contact areas, each contact area being located to engage with one or more of the assigned immobilization points, and a dimension calculation module operable to select, from a cast template database, a cast template having contact areas that most closely correspond to the mapping of contact areas.

In yet another aspect, there is provided a kit of parts for an orthopedic cast, comprising a substantially rigid inside wrist portion having a palm support adapted to engage with a palm of a wearer and an inside arm guard adapted to engage with the inside of a forearm of a wearer. The cast further comprises a substantially rigid outside wrist portion having a back hand guard for engaging the back of the wearer's hand and an outside arm guard for engaging the outside of the forearm of the wearer. The inside wrist portion and outside wrist portion being adapted to be secured to the wrist of a wearer to immobilize the wearer's wrist.

In yet another aspect, there is provided a method of applying the cast to a wearer. The method comprises aligning the palm support with the palm of a wearer, aligning the inside arm guard with the inside of the wearer's forearm, aligning the back hand guard with the back of the wearer's hand, aligning the outside arm guard with the forearm of the wearer, and securing each of the inside wrist portion and the outside wrist portion to the arm of the wearer.

This Summary is provided to introduce a selection of concepts in a simplified form, examples of which are described in the Detailed Description. This Summary is not intended to identify key features or essential features of the claimed subject matter, nor is it intended to be used as an aid in determining the scope of the claimed subject matter.

### DISCLOSURE OF THE INVENTION

Example embodiments of the proposed casts, kits, and related systems and methods will now be described by way of example only with reference to the accompanying drawings.

### BRIEF DESCRIPTION OF THE DRAWINGS

FIG. 1 is a perspective view of a cast on a wearer's wrist;

FIG. 2 is a perspective view of the cast of FIG. 1 removed from the wearer's wrist;

FIG. 3 is a side view of the cast of FIG. 1 on a wearer's wrist;

FIG. 4 is a side view of the cast from the perspective of FIG. 3 removed from the wearer's wrist;

FIG. 5A is a cross-sectional view along line A-A of the cast of FIG. 4;

FIG. 5B is a cross-sectional view along line B-B of the cast of FIG. 4;

FIG. 5C is a cross-sectional view along line C-C of the cast of FIG. 4;

FIG. 5D is a cross-sectional view along line D-D of the cast of FIG. 4;

FIG. 5E is a cross-sectional view along line E-E of the cast of FIG. 4;

FIG. 6A is a perspective view of the upper portion of the cast of FIG. 1;

FIG. 6B is a perspective view of the upper portion of the cast similar to FIG. 6A;

FIG. 6C is a perspective view of the skin-engaging surface of the upper portion of the cast of FIG. 1;

FIG. 7A is a perspective view showing the skin-engaging surface of the lower portion of the cast of FIG. 1;

FIG. 7B is a rear perspective view of the lower portion of the cast rotated with respect to FIG. 7A to show a side view of the palm support;

FIG. 7C is a side perspective view of the lower portion of the cast rotated with respect to FIG. 7A to show a side view of the inside arm guard;

FIG. 8A is an overhead view of the inside of a wrist showing example immobilization points;

FIG. 8B is a side view of the finger side of the wrist of FIG. 8A showing additional example immobilization points;

FIG. 8C is an overhead view of the back side of the wrist of FIG. 8A showing additional example immobilization points;

FIG. 8D is a side perspective side view of the thumb side of the wrist of FIG. 8A showing additional example immobilization points;

FIG. 8E is a musculoskeletal diagram of the back side of a wrist showing other example immobilization points;

FIG. 8F is a musculoskeletal diagram of the inside of the wrist similar to FIG. 8E showing other example immobilization points;

FIG. 9A is an overhead view of the inside of a wearer's wrist similar to that of FIG. 8A wherein the user is wearing a cast;

FIG. 9B is a side perspective view of the finger side of the wrist similar to that of FIG. 8B wherein the user is wearing a cast;

FIG. 9C is an overhead view of the back side of the wrist similar to that of FIG. 9B wherein the user is wearing a cast;

FIG. 9D is a perspective side view of the thumb side of the wrist similar to that of FIG. 8D wherein the user is wearing a cast;

FIG. 10 is an overhead view of a cast on the inside of a wearer's wrist wherein the cast immobilizes the wrist from pronation and supination;

FIG. 11 is an overhead view of a cast on the outside of a wearer's wrist wherein the cast immobilizes the wrist from pronation and supination;

FIG. 12 is a side view of a cast on the wearer's wrist wherein the cast immobilizes the wrist from flexion and dorsiflexion;

FIG. 13 is an overhead view of a cast on the outside of a wearer's wrist wherein the cast immobilizes the wrist from adduction and abduction;

FIG. 14 is an overhead view of a cast on the inside of a wearer's wrist wherein the cast immobilizes the wrist from adduction and abduction;

FIG. 15 is a system diagram of an example cast model generator;

FIG. 16 is a flow diagram of an example process for producing a cast fitted to a wearer;

FIG. 17 is a flow diagram of an example process for selecting a cast from a cast template database; and

FIG. 18 is a flow diagram of an example method of attaching a cast to a wearer.

### DETAILED DESCRIPTION OF THE DRAWINGS

It will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures and components have not been described in detail so as not to obscure the example embodiments described herein. Also, the description is not to be considered as limiting the scope of the example embodiments described herein.

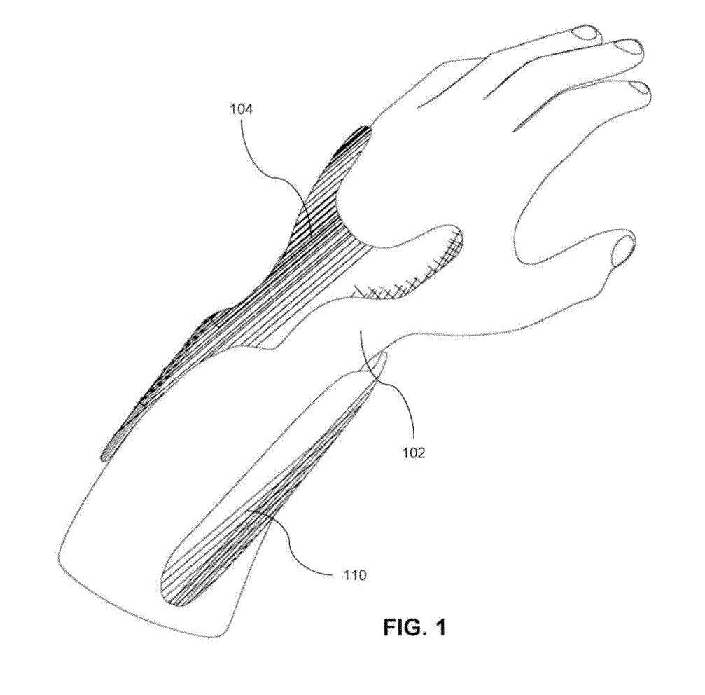

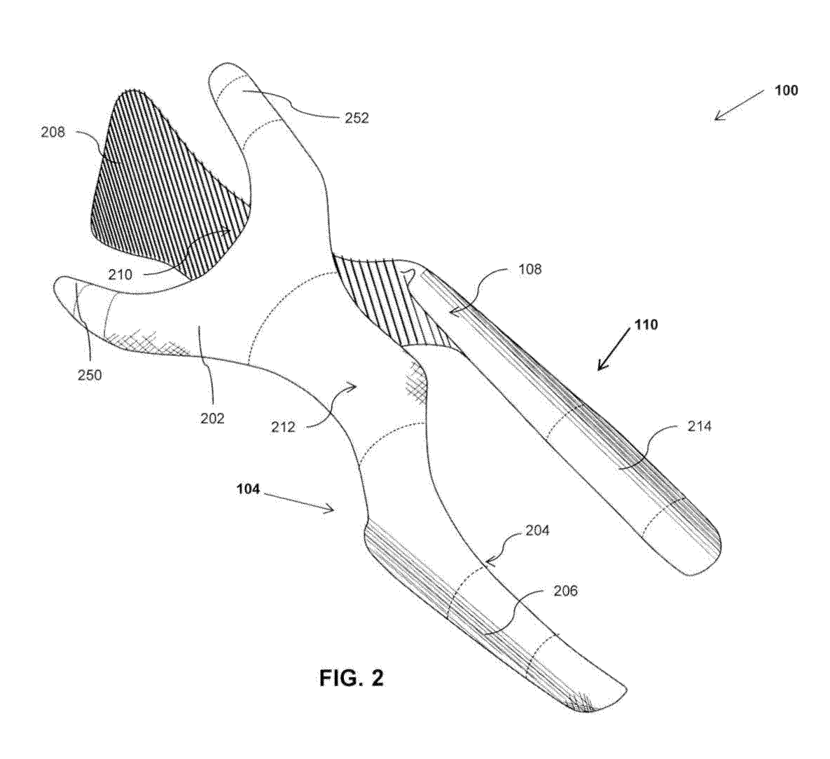

Turning to FIG. 1 and FIG. 2, an example of a cast 100 for immobilizing a left hand is provided. The cast 100 is shown on a wrist 102 of a wearer in FIG. 1 and removed from the wrist 102 in FIG. 2. The cast 100 comprises a substantially rigid inside wrist portion 110 and a substantially rigid outside wrist portion 104.

It will be appreciated that the term “immobilizing” may include complete immobilization, substantial immobilization, or a lesser degree of restricted movement. By way of example, a wrist may be considered to be immobilized in the context of a particular injury if the wrist is sufficiently immobilized to enable healing of the injury.

The inside wrist portion 110 extends from a palm support 208, and is formed to engage the inner side of the wrist 102 of the wearer. The outside wrist portion 104 extends from a back hand guard 202 formed to engage the back of the hand and is formed to engage the outer side of the wrist at an outside arm guard 206.

Each of the inside wrist portion 110 and outside wrist portion 104 comprise contact areas, which are configured to be positioned on the wearer. The inside wrist portion 110 and outside wrist portion are substantially rigid such that some or all of the contact areas are held immobile with respect to each other.

The inside wrist portion 110 comprises an inside arm guard 214 to engage the inside of the forearm. The inside arm guard 214 provides lateral support to the inside wrist portion 110. The palm support 208 extends inward toward the wearer's palm and comprises a palm contact 402, for contacting the skin on the palm.

The cast 100 may be formed from various materials known in the art including polymers and composites.

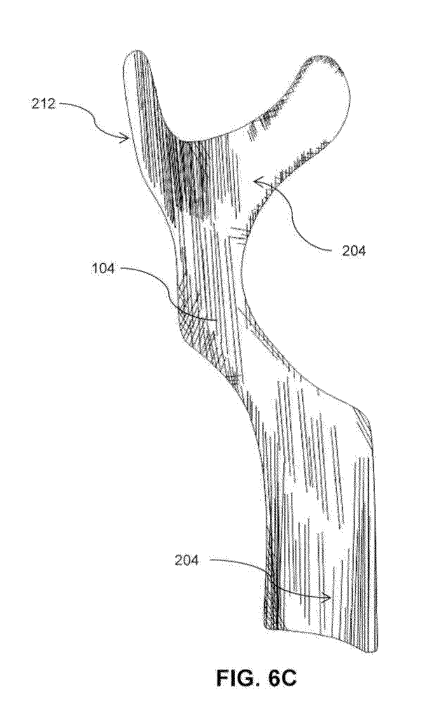

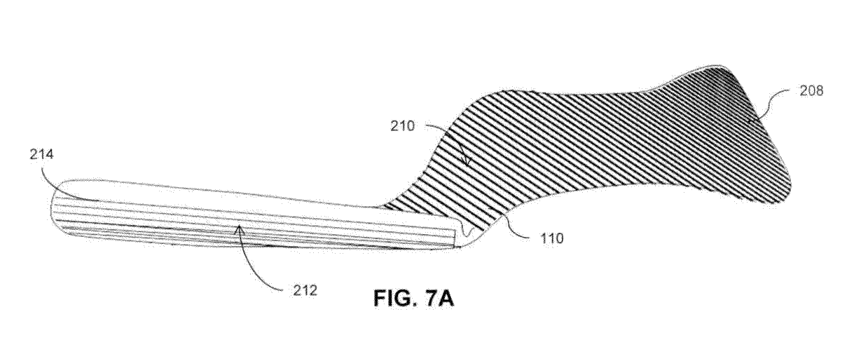

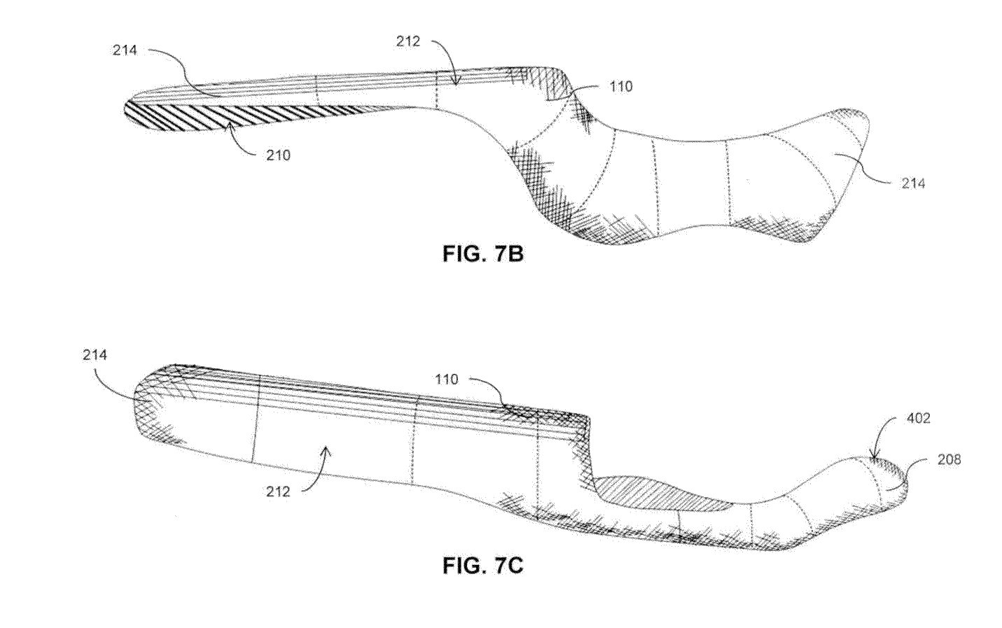

Turning to FIGS. 7A to 7C, the inside wrist portion 110 comprises a first skin-facing surface 210 and a first outer surface 212. Predetermined areas of the first skin-facing surface 210, which are referred to herein as contact areas, are contoured to engage the skin of a wearer. For example, a palm contact 402 of the palm support 208 engages the skin on the palm of the wearer's hand and a contact area on the inside arm guard 214 of the inside wrist portion 110 at least partially contacts the skin on the inside of the forearm.

Portions of the skin-facing surface 210 may optionally be raised with respect to any contact areas so as to be spaced apart from the skin of the wearer to promote airflow and transpiration between the wearer's arm 102 and the cast 100. An intermediary layer, for example a breathable membrane, may optionally be placed between certain portions of the skin of the wearer and the cast to improve transpiration of the skin. In another example, an intermediary layer is placed only at certain portions between the wearer and the cast 100. For example, an intermediary layer may be placed between the cast 100 and the wearer in all regions except for the contact areas.

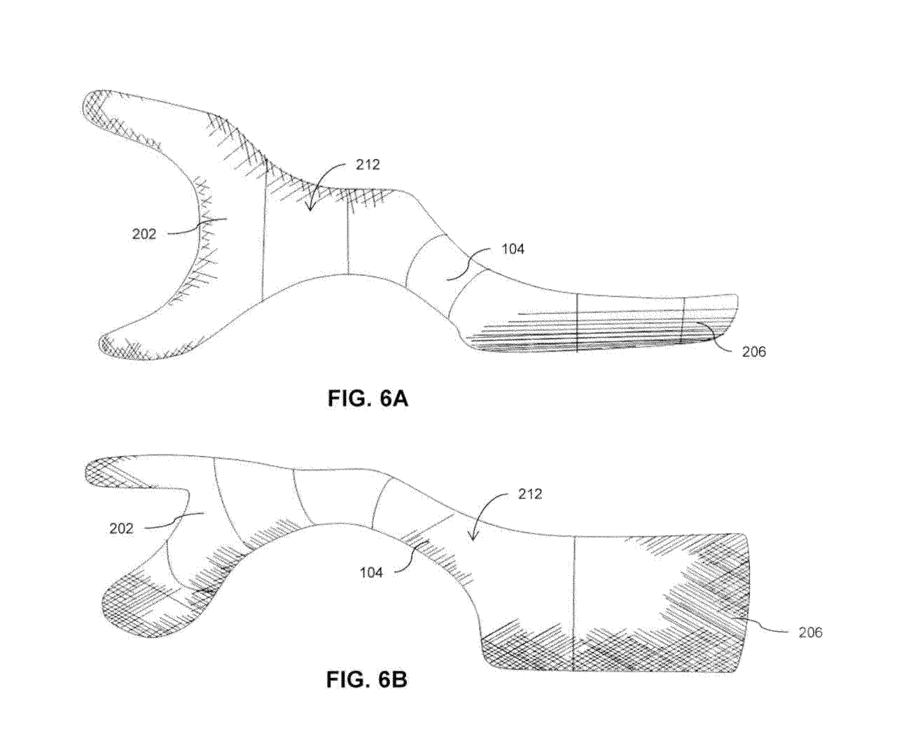

Turning to FIGS. 6A to 6C, the outside wrist portion 104 comprises a second skin-facing surface 204 and a second outer surface 212. Predetermined contact areas on the second skin-facing surface 204 are contoured to engage the skin. The outside wrist portion 104 further comprises the back-hand guard 202 to engage the back portion of the hand, as shown in FIG. 1. As is shown in FIG. 2, the back-hand guard 202 may comprise a pair of forwardly extending hand supports 250, 252, each of which include a contact area to contact the wearer's skin on forward portions of the back of the wearer's hand. The outside arm guard 206 of the outside wrist portion 104 comprises contact areas to contact the skin on the outside of the upper arm, similarly to the inside arm guard 214 on the inside of the upper arm.

As will be appreciated from FIGS. 1 and 2, the outside arm guard 206 and the inside arm guard 214 cooperate to retain the forearm of the wearer while the back hand guard 202 and palm support 208 cooperate to retain the hand of the wearer. As shown in FIGS. 1 and 2, the outside arm guard 206 and the inside arm guard 214 are located on generally opposing sides of the forearm.

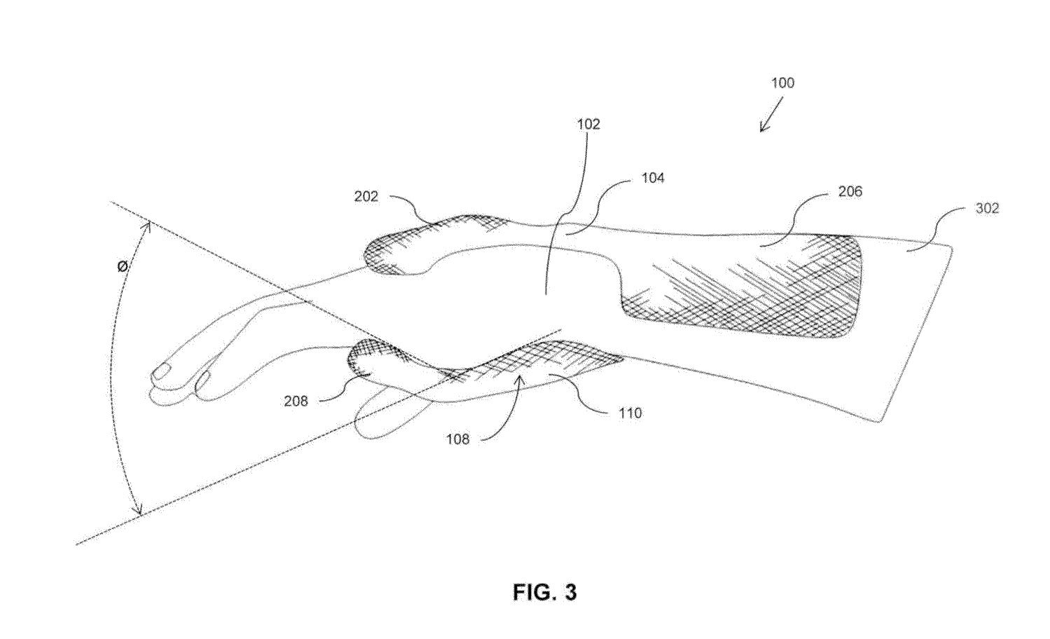

Referring now to FIG. 3, the cast 100 is shown on a wrist 102 from the small finger side of the hand. When the cast 100 is worn, the palm support 208 is angled, as represented by angle ø towards the palm of the wearer and is in contact with at least a portion of the wearer's palm. Angle ø is the angle between a plane defined substantially by the palm support 208 and a plane defined substantially by the inside wrist portion that extends along the wrist, as is shown in FIG. 3. Angle ø may vary between, for example, 15 degrees and 45 degrees. More specifically, angle ø may be between about 25 and 35 degrees. For example, ø0 may be about 30 degrees to support the palm and enable the wrist to heal with the wrist in a substantially neutral position. It will also be appreciated from the example of FIG. 3 that the back hand guard 202 extends across the back of the wearer's hand but does not cover the wearer's knuckles, allowing the wearer a range of finger motion, which may be desirable for certain wrist injuries.

The outside wrist portion 104 extends from the back-hand guard 202 to the outside arm guard 206. As is shown in FIG. 3, the outside wrist portion 104 curls around the wearer's wrist 102 such that one or more contact areas on the second skin-facing surface 204 of the outside arm guard 206 engage the arm 302 of the wearer. Similarly, the inside wrist portion 110 extends from the palm support 208 to the inside arm guard 214 and curls around the wearer's wrist such that one or more contact areas on the inside arm guard 214 contacts a portion of the side of the wearer's arm 302 or wrist 102 or even a portion of the back side of the wearer's arm 302. This arrangement of the cast provides an outside wrist portion 104 that substantially opposes an inside wrist portion 110 to substantially immobilize a wrist.

As will be appreciated from the examples of FIGS. 1 and 2, the inside wrist portion 110 and the outside wrist portion 104 of the cast 100 are separate components. Each of inside wrist portion 110 and the outside wrist portion 104 can be fixed independently to the skin of the wearer. For example, the outside wrist portion 104 and the inside wrist portion 110 may be fixed to the skin of the wearer using an adhesive. Preferably, the adhesive is a medical-grade biocompatible adhesive. The adhesive may be applied specifically to the skin-facing surfaces 210 and 204, and more specifically to the contact areas of the cast 100 to render the contact points of the cast 100 substantially immobile with respect to the wearer's skin. One group of biocompatible adhesives that may be used comprises silicone-based adhesives. Other appropriate biocompatible adhesives such as acrylics, urethanes, cyanoacrylates, and epoxies may also be used. The biocompatible adhesive may comprise a spray-on adhesive, a ribbon or patch adhesive, a liquid adhesive, or other appropriate adhesives including double-sided tape. A cast may comprise a patch of adhesive with a removable covering that is removed to expose the adhesive before installing the cast on a wearer.

Other methods of attaching the inside wrist portion 110 and the outside wrist portion 104 to the wearer's arm may be used as a substitute for, or in addition to, a biocompatible adhesive. Optionally, the inside wrist portion 110 may be secured to the outside wrist portion 104 to secure the wearer's wrist. For example, the inside wrist portion 110 may be secured to the outside wrist portion 104 using adjustable or elastic straps, which may be tightened about a user's arm to secure the cast 100. By way of example, the inside wrist portion 110 may be secured to the outside wrist portion 104 by means of a belt comprising hook and loop fastener. It will be appreciated that various other methods of attaching the inside wrist portion to the outside wrist portion may be used.

In an example where the inside wrist portion 110 and the outside wrist portion 104 are fixed to a wearer's arm using a medical grade adhesive, the cast 100 may be removed from the wearer's arm using an appropriate biocompatible adhesive remover. It will be appreciated that the cast may be removed and re-applied a number of times, for example, using a medical grade adhesive.

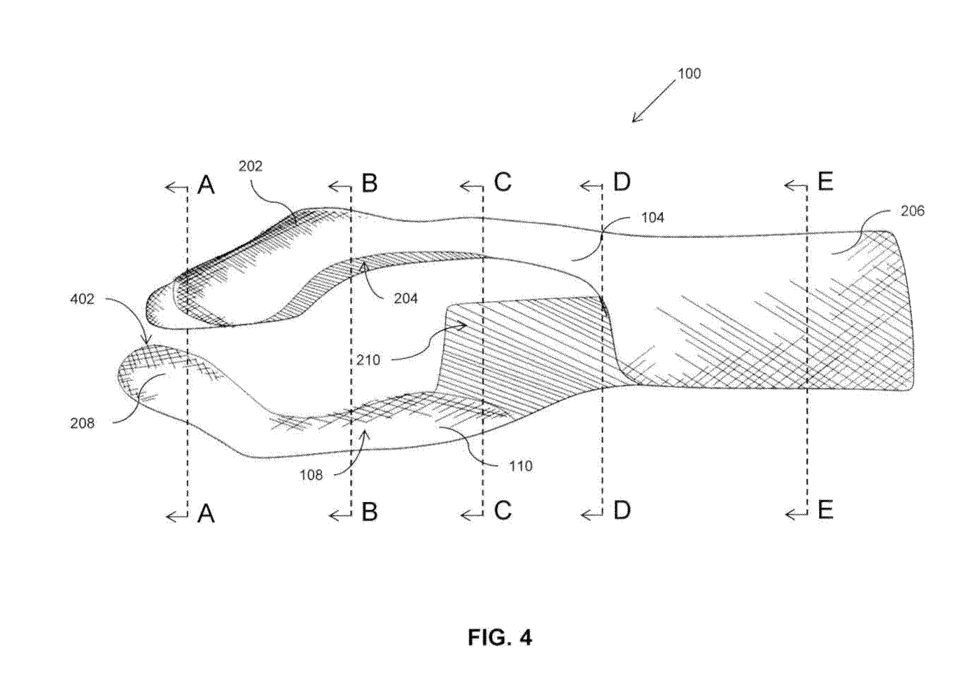

Referring now to FIG. 4, the cast 100 of FIG. 3 is removed from a wearer's arm 302. It can be seen that the second skin-facing surface 204 in the region of the back hand guard 202 is concave to receive the curved surface of the back side of a wearer's hand. Furthermore, the palm contact 402 of the palm support 208 is convex such that the palm contact fits comfortably into the palm of the wearer's hand to immobilize the wearer's hand in cooperation with the back hand guard 202.

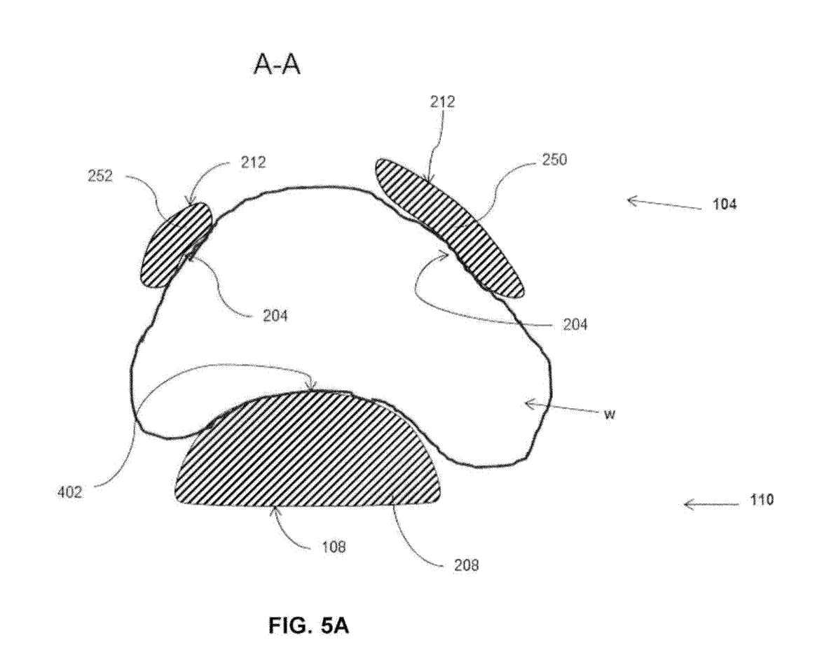

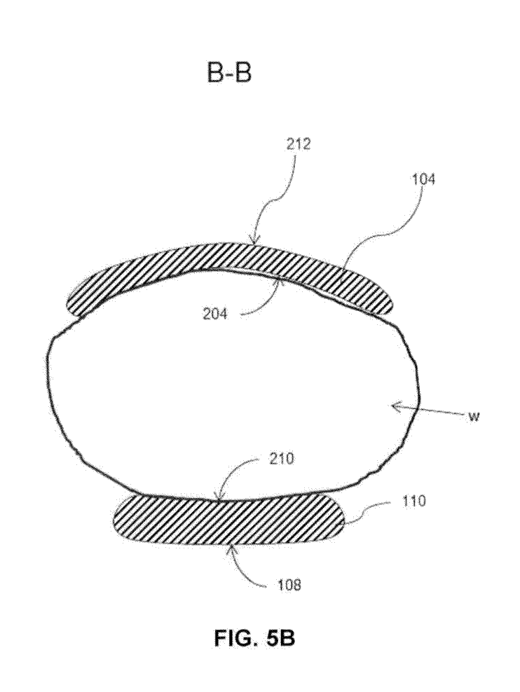

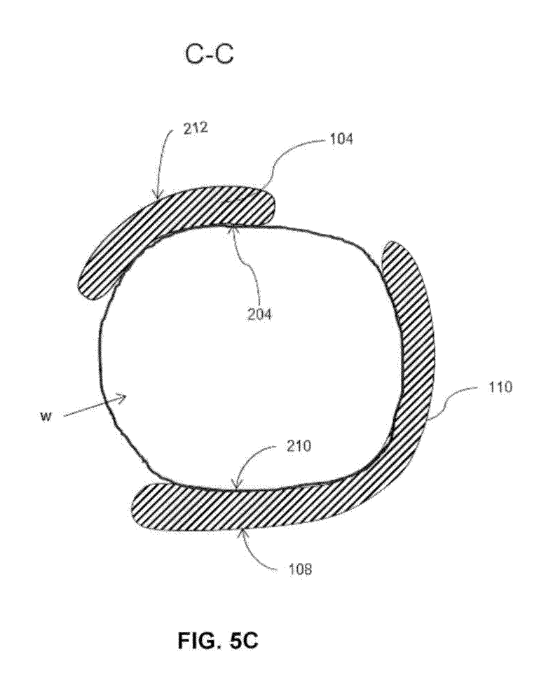

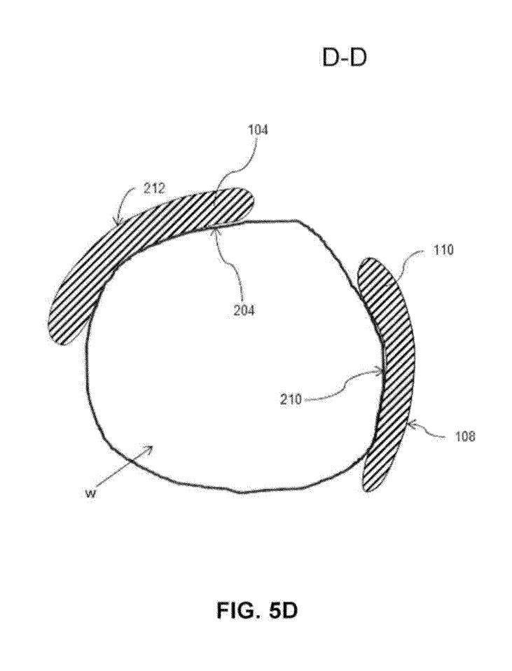

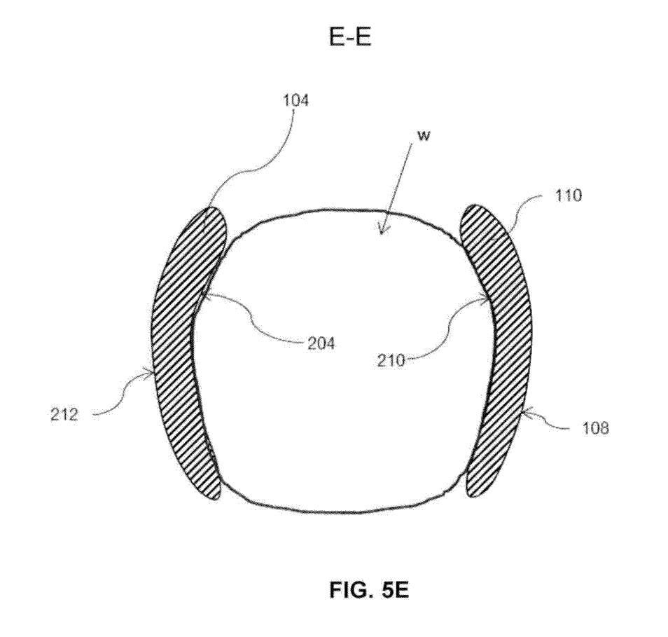

FIGS. 5A through 5E show progressive cross sections of the cast 100 taken in the direction moving from the palm support 208 to the inside arm guard 214. As shown in these figures, the relative positioning between the inside arm portion 110 and the outside arm portion 104 is substantially in contact along the length of the hand, wrist, and arm of a wearer. As is shown, the position of the cast on the wrist depends on the location along the length of the hand, wrist, and arm, which is represented by w in each of FIGS. 5A through 5E.

Turning now to the section of FIG. 5A, the outside wrist portion 104, as outlined above, has a concave second skin-facing surface 204 to receive the hand of the wearer. The forwardly extending hand supports 250, 252 are shown separated in the A-A section. The second outer surface 212 is concave such that the cast 100 moulds to the contour of the wearer's hand. Conversely, the palm contact 402 is convex such that it contacts the palm substantially uniformly in the ball of the palm. Specifically, the palm contact 402 is convex in both the longitudinal direction, which is aligned with the axis of the wearer's forearm, and in the transverse direction, which is defined as the direction transverse to the wearer's forearm.

The pair of forwardly extending hand supports 250, 252 cooperates with the palm contact 402 of the palm support 208 of the inner wrist portion 110 to secure the hand. The wearer's hand is secured between the back hand guard 202 and the palm support 208 to prevent motion in the wearer's wrist 102 that would be deleterious to the healing process of the fracture or other injury.

Referring now to FIG. 5B, a section of the cast 100 along B-B of FIG. 4 is shown. As can be seen from the section of the outside wrist portion 104, and similarly to the section of FIG. 5A, the second skin-facing surface 204 is concave and the second outer surface 212 is convex. The second skin-facing surface 204 is contoured to accommodate the region about the hand of the wearer. It will be appreciated that, in contrast to the palm contact 402 of the palm support 408, the first skin-facing surface 210 is concave to comfortably receive the contours of the base of the wearer's palm.

FIG. 5C shows the cross section C-C, which is taken approximately longitudinally midway on the cast 100 at about the wearer's wrist. Section C-C is at a location substantially consistent with a wrist fracture. As is apparent from the figure, the outside wrist portion 104 curves around the wrist 102 to the little finger side of the arm 302 at the outside arm guard 206. Correspondingly, the inside wrist portion 110 curves around the wrist 102 to the thumb side of the arm 302 at the inside arm guard 214. In other words, the inside wrist portion 110 and the outside wrist portion 104 curve around the arm 302 in a clockwise direction when taking progressive sections beginning at the hand region of the cast 100 and moving longitudinally to the arm region of the cast. 100.

FIG. 5D, along section D-D of FIG.4, shows the inside wrist portion 110 and the outside wrist portion 104 continuing the clockwise curve as progressive sections are taken towards the inside arm guard 214 and outside arm guard 206 of the cast 100. FIG. 5E, along E-E of FIG. 4, shows the cast in the region of the inside arm guard 214 and the outside arm guard 206, which are located on approximately the inside of the forearm 302 and the outside of the forearm 302, respectively.

The contours of the example outside wrist portion 104 are shown in FIGS. 6A and 6B. The second skin-contacting surface 204 is contoured to comfortably receive the wearer's arm 302. Referring to FIG. 6C, some, or all, of the second skin-facing surface 204 on the outside arm guard 206 and the back hand guard 202 may be contoured to comfortably contact the skin on the wearer's arm 302 and hand, respectively.

Referring again to FIGS. 7A through 7C, which show the form of the example inside wrist portion 110, at least a portion of the first skin facing surface 210 is contoured to receive the arm 302 of a wearer. For example, the inside wrist portion 110 comprises a palm contact area 402, an inner wrist contact area, and a first arm contact area on the inside arm guard 214. Each of the contact areas is secured to the skin the user, for example, using a biocompatible adhesive.

It is also recognized that immobilizing specific points or areas in the vicinity of a fracture with respect one another can, in some cases, be sufficient to effectively immobilize a fracture to enable the fracture to heal. It is therefore not necessary to completely envelop the area around a fracture with a cast. These points in the vicinity of the fracture may be referred to interchangeably as “immobilization points” or “immobilization areas”. For simplicity, the term “immobilization points” as used herein encompasses both immobilization points and immobilization areas.

By way of example, a cast 100 for a wrist fracture that immobilizes predetermined immobilization points in the vicinity of a user's wrist may be effective in immobilizing the wrist. The cast 100 comprises contact areas which can be secured to each of the immobilization points. The contact areas on the cast 100 are interconnected by substantially rigid portions of the cast 100 to prevent relative motion between the immobilization points in the vicinity of the user's wrist.

In an example embodiment, the cast may also comprise non-rigid portions, for example resilient or flexible portions, in addition to the rigid portions which connect the contact areas.

A cast that immobilizes only predetermined immobilization points with respect to one-another may contribute to the mitigation of one or more of the disadvantages associated with traditional casts. For example, a cast that only makes contact with predetermined immobilization points of a wearer of a cast may reduce the dermatological impact of the cast in the areas where the cast is not in direct contact with the skin. Such a cast may also be less cumbersome, as less material is required to achieve immobilization of an injury.

The contact areas may be the only areas of the cast that are required to contact the skin to sufficiently immobilize the injury. As such, areas of the cast that do not constitute contact areas may, but don't necessarily, contact the wearer's skin.

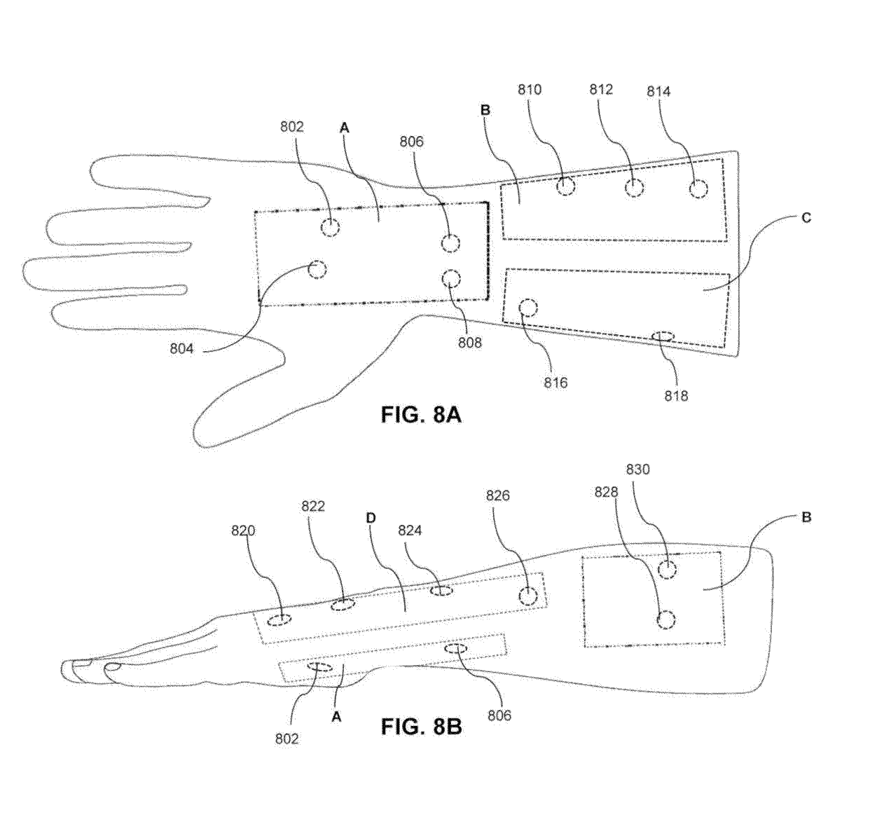

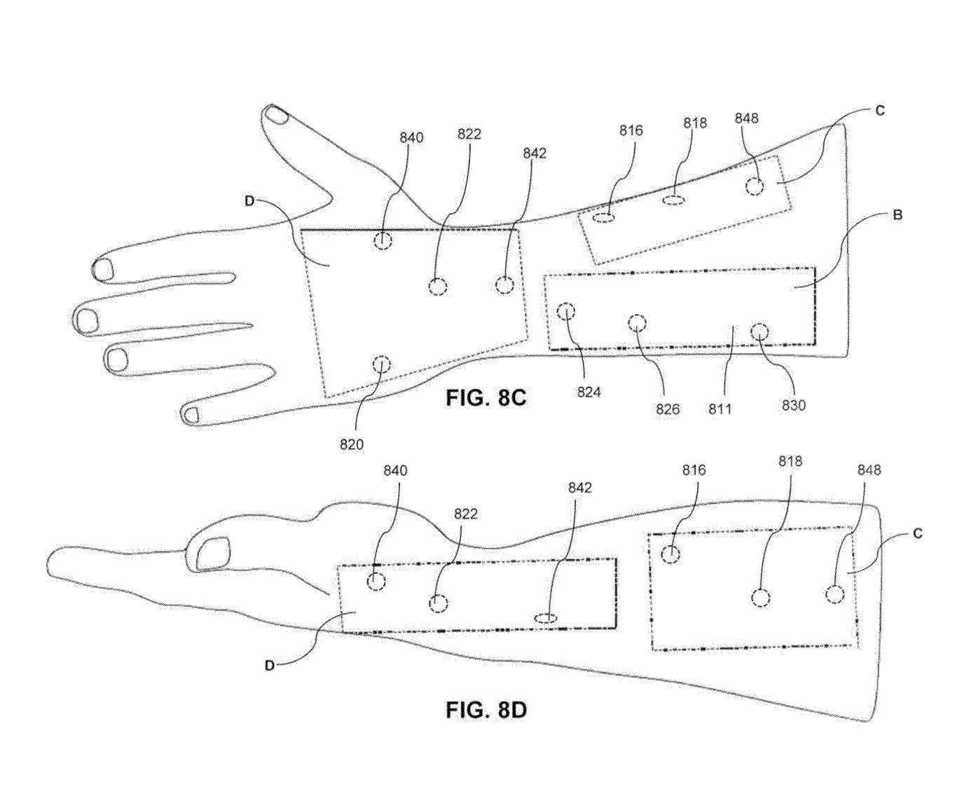

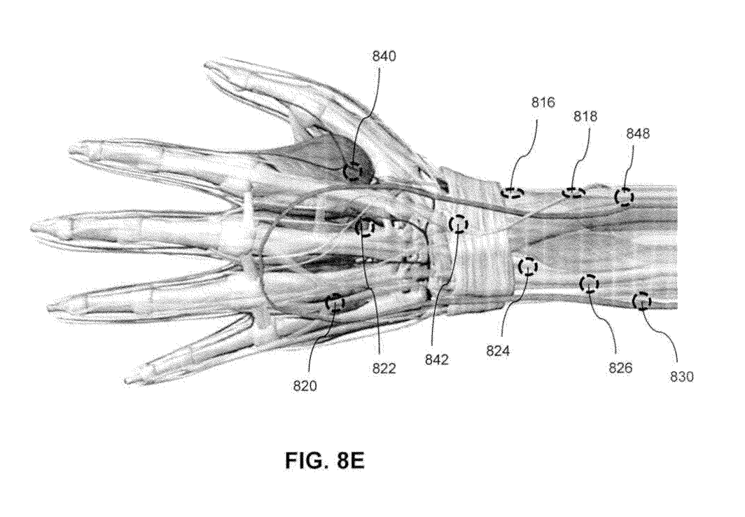

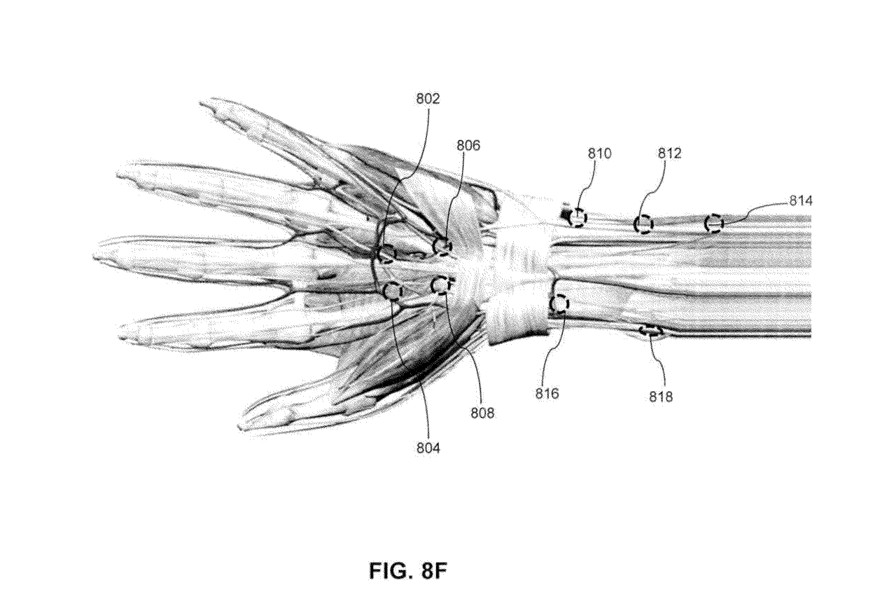

Referring now to FIG. 8A through FIG. 8D, a wearer's hand is shown from various perspectives. From each of these perspectives, a number of example immobilization can be seen on the wearer's hand. It will be appreciated that other immobilization points may be used in place of, or in addition to, the example immobilization points outlined herein. It will also be appreciated that the immobilization points may be circular, ovoid, or any other regular or irregular shape.

In an example, at least one immobilization point is provided in each of immobilization point areas A through D as shown in FIGS. 8A through 8D. It will be appreciated that, as shown in FIGS. 8A though 8D, more than one immobilization point may be provided within an immobilization area. In the example of FIGS. 8A through 8D, an immobilization point in each of the immobilization areas may be sufficient to immobilize a wrist.

It will also be appreciated that the immobilization areas of FIGS. 8A through 8D are example immobilization areas only and are intended to show an example of the regions of the wrist, hand, and arm on which immobilization points may be located. Additional, fewer, or substitute immobilization point areas may be used.

Specifically, FIGS. 8A and 8B show example palm immobilization points 802 and 804 which can be engaged by the palm contact area 402, and wrist immobilization points 806, 808, which can be engaged by a lower portion of the palm support 208. Inner arm immobilization points 810, 812, and 814 can be engaged by the outside arm guard 206. Example inner arm immobilization points 816, and 818 can be engaged by the inside arm guard 214.

FIGS. 8C and 8D show example immobilization points on the back side of the hand, specifically back hand immobilization points 820, 822, 840, and 842, can be engaged by the outside wrist portion 104. Specifically, immobilization points 820 and 840 can be engaged by the forwardly extending hand supports 820 and 840, respectively. In addition to immobilization points 816 and 818, immobilization point 848 can be engaged by the inside arm guard 214.

Although specific example immobilization points are provided in FIGS. 8A through 8D, it will be appreciated that the specific immobilization points used may differ depending on various parameters including the size of the wrist, the type of injury that the wearer has sustained, and the degree to which immobilization is required. As such, the cast 100 may be adapted to engage with additional immobilization points, substitute immobilization points, or fewer immobilization points depending on the degree of immobilization required.

FIG. 8E and FIG. 8F show a set of example immobilization points overlaid on the musculoskeletal structure of the wrist. Specifically, FIGS. 8E and 8F shows the immobilization points outlined above with reference to FIGS. 8A through 8D. Many of the immobilization points, including 820, 822, and 840, are selected to be in a structural location, for example, a location that is proximal to a bone or where two bones meet. If the immobilization points shown in FIG. 8E and FIG. 8F are held immobile relative to each other, the wrist may be sufficiently immobilized.

Again, it will be appreciated that immobilization points in addition to those outlined in FIGS. 8A through 8F may be used. Similarly, it will be appreciated that not every one of the immobilization points outlined in FIGS. 8A through 8F is required for each type of injury. As such, a cast may comprise additional, or fewer, contact areas than the number of immobilization points presented in FIGS. 8A through 8F.

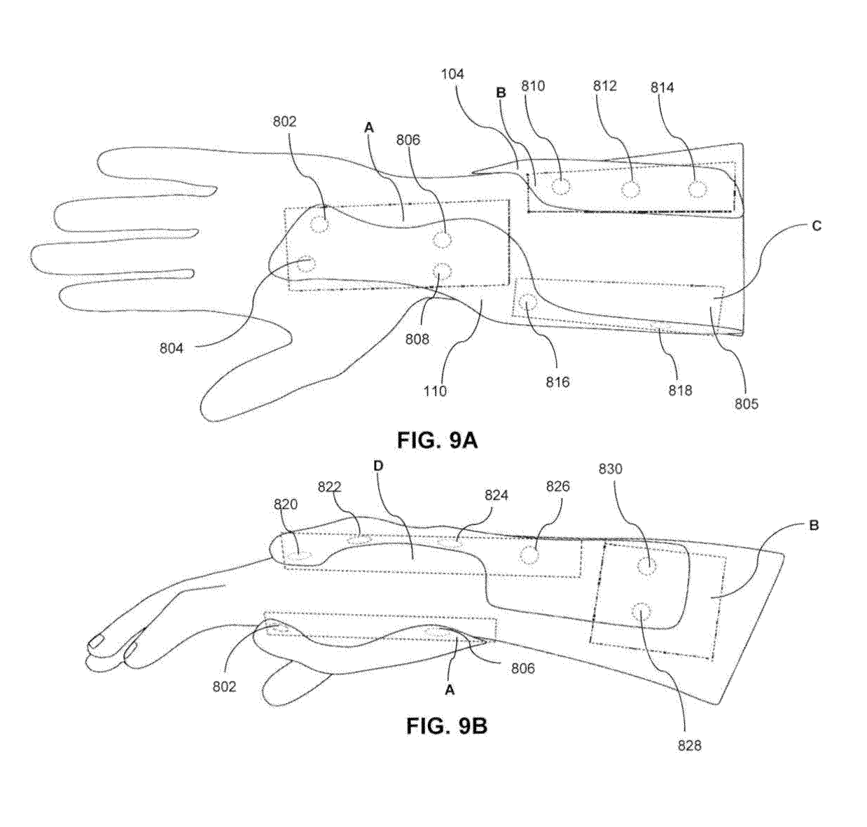

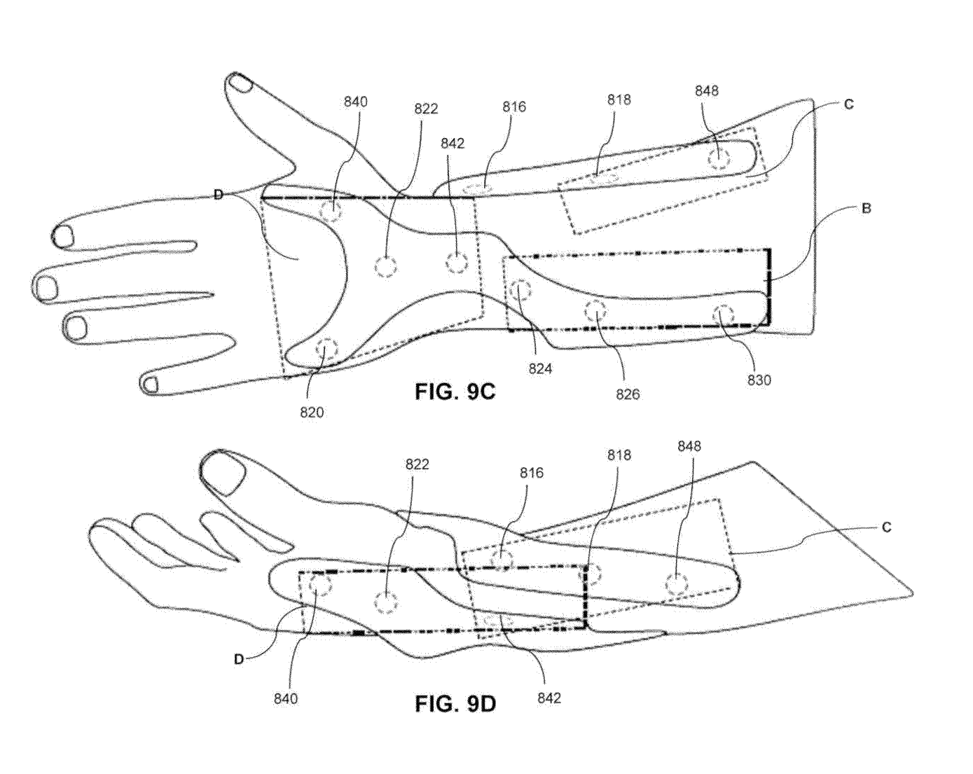

Referring now to FIG. 9A to FIG. 9D, the cast 100 is shown being worn by a user from the same perspectives as FIG. 8A to FIG. 8D, respectively. As outlined above, contact areas on the skin-facing surface of the cast 100 are contoured to engage at least each of the immobilization points, for example, those outlined in FIGS. 8A through 8D.

FIG. 9A shows the wearer wearing the cast 100 with a contact area located on the palm contact 402 overlaid on the first immobilization point 802 on the wearer's hand. A contact area may engage two or more immobilization points. For example, the palm contact area 402 may engage both of immobilization points 802 and 804.

Specifically, FIGS. 9A and 9B show the cast 100 overlaid on a hand whereby the palm support 208 is contoured to engage example palm immobilization points 802 and 804, and wrist immobilization points 806, 808. The outside arm guard 206 engages inner arm immobilization points 810, 812, and 814, whereas the inside arm guard 214engages immobilization points 816 and 818. The outside wrist portion 104 engages the back hand immobilization points 820, 822, 840, and 842. Specifically, the forwardly extending hand supports 820 and 840 engage points 820 and 840, respectively. Immobilization point 848 is engaged by the inside arm guard 214.

As such, when the contact areas are secured to the immobilization points, the wrist 102 is held substantially immobile between the inside wrist portion 110 and the outside wrist portion 104.

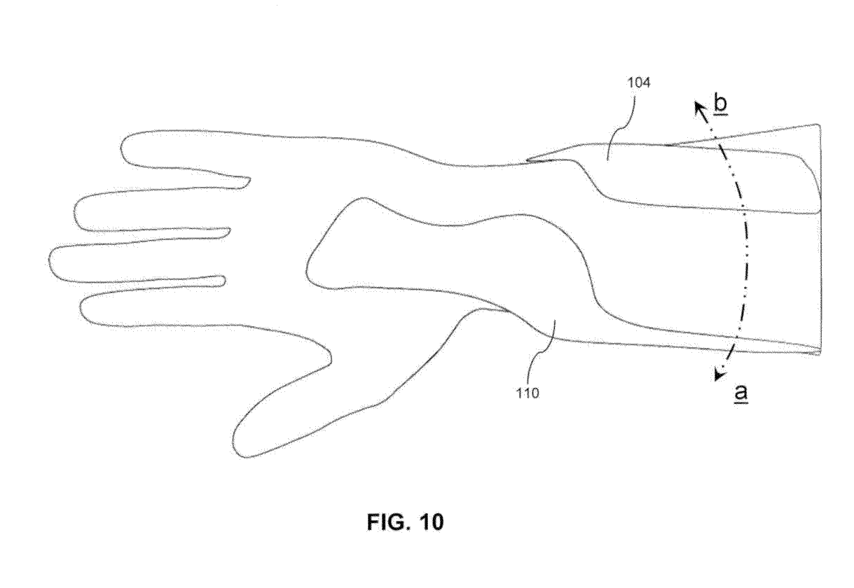

FIGS. 10 through 14 demonstrate various wrist movements that a cast 100 as described herein may restrict or inhibit completely. Specifically, by securing contact areas on the cast 100 to the immobilization points, the substantially rigid inside wrist portion 110 and outside wrist portion 104 of the cast reduce relative motion between each of the immobilization points to substantially immobilize the wrist 102.

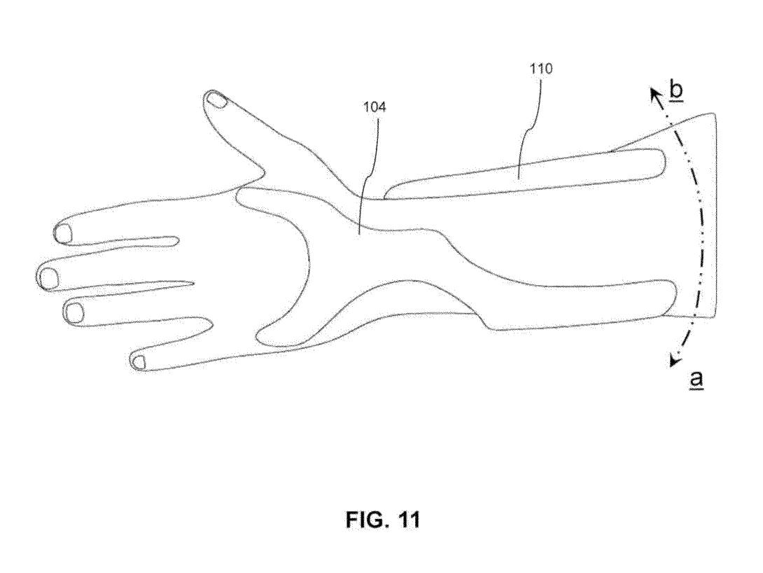

Turning now to FIGS. 10 and 11, the cast secures the wrist 102 to prevent pronation at direction a and supinating at direction b. When a pronating motion is attempted with the wrist 102, as indicated by direction a, the immobilization points 802 through 824 in contact with contact areas 902 through 924 are held substantially immobile, thereby suppressing the pronating motion. Similarly, when a supinating motion is attempted with the wrist 102, as indicated by direction b, the supinating motion is suppressed, thereby maintaining the wrist in a substantially immobile position.

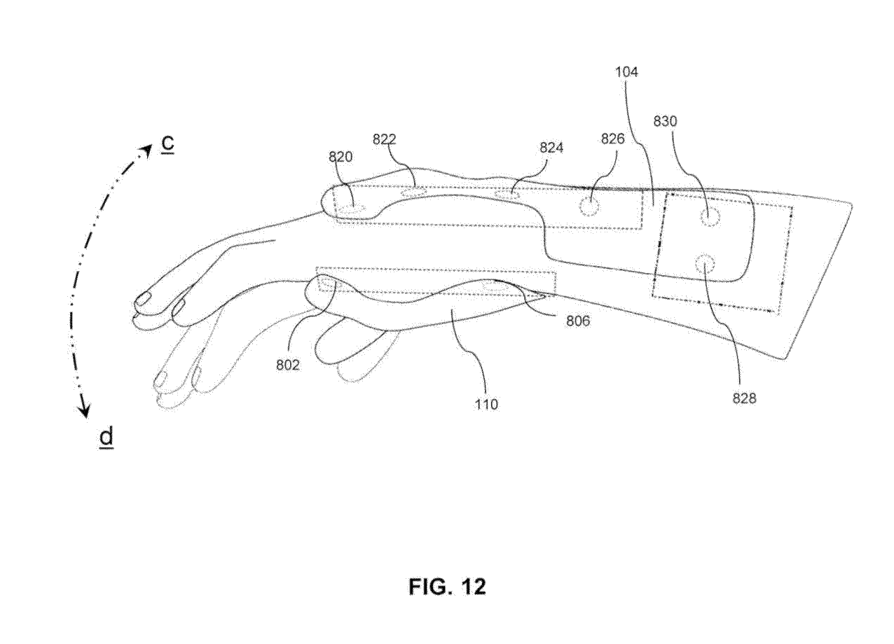

Referring to FIG. 12, the immobilization points and contact areas shown in each of FIGS. 8A through 8D and FIGS. 9A through 9D cooperate to prevent flexion, as outlined by direction c and dorsiflexion, as outlined by direction d. As such, wrist motion toward and away from the palm is suppressed, which also contributes to immobilizing the wrist 102.

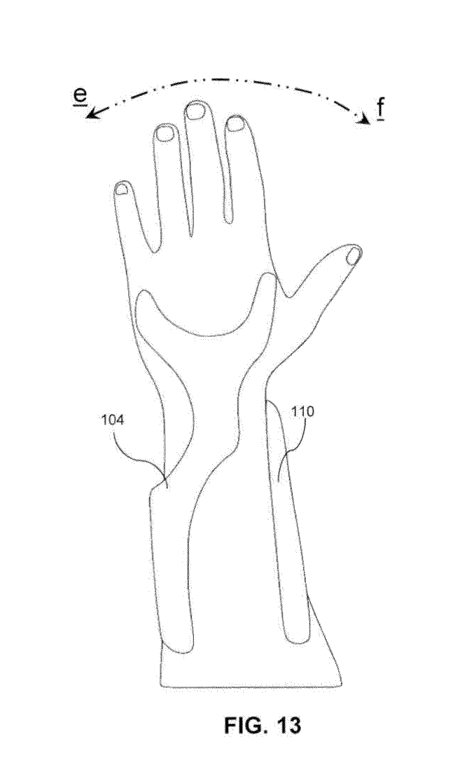

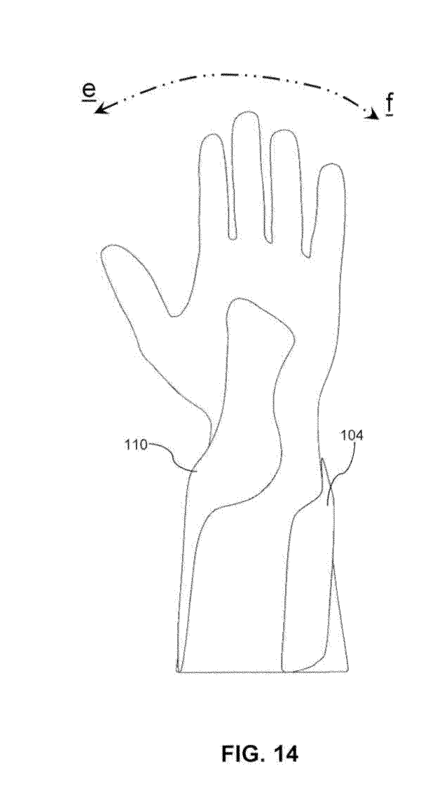

FIGS. 13 and 14 demonstrate that is a the immobilization points and contact areas shown in each of FIGS. 8A through 8D and FIGS. 9A through 9D also cooperate to prevent adduction and abduction, as indicated by directions e and f, respectively. Therefore, motion toward and away from the thumb side of the hand is suppressed, which assists in immobilizing the wrist 102.

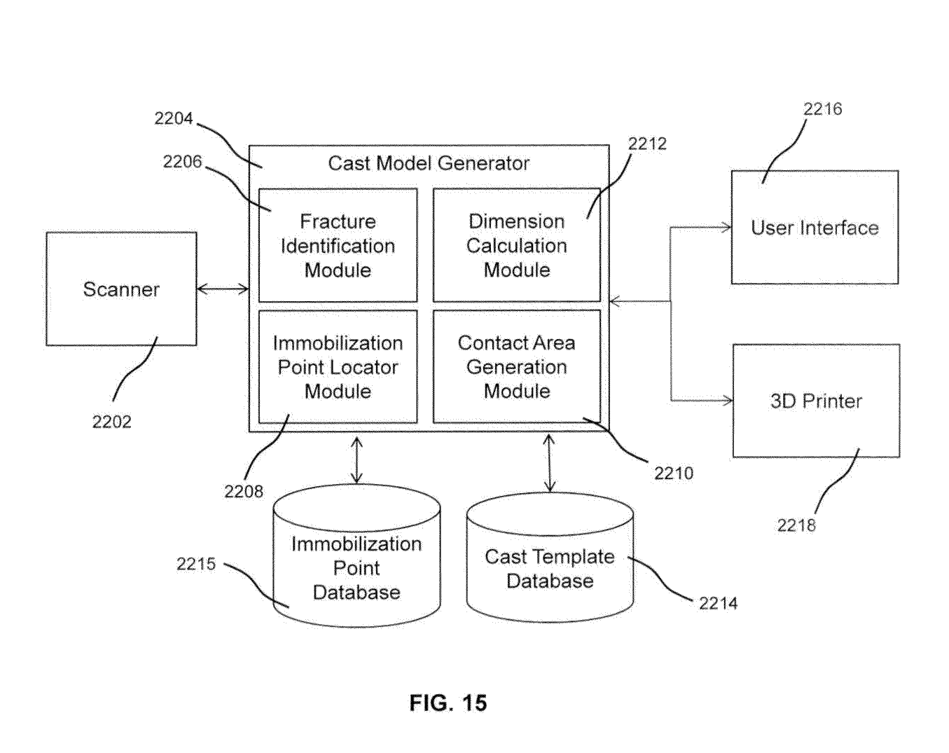

Referring now to FIG. 15, a system for producing a model of a cast is provided. The system comprises a cast model generator 2204, which is linked to a scanner 2202, a cast template database 2214, an immobilization point database 2215, a user interface 2216, and a programmable fabrication unit, which is embodied by a three dimensional printer 2218 in the example of FIG. 15.

The scanner 2202, cast model generator 2204, display 2212, and 3D printer 2218 of FIG. 15 each comprises, or is linked to, a memory and a processor. The user interface 2216 may comprise a display and an input device. For example, the input device may comprise a mouse, a keyboard, a stylus, a touchscreen, or other suitable device. In an example embodiment, the cast model generator 2204 is a computing device.

The scanner 2202 may comprise an x-ray scanner, a magnetic resonance imaging scanner, or another suitable scanner for imaging anatomy. For example, the scanner 2202 may comprise an x-ray computed tomography scanner. The scanner 2202 is operable to scan an affected area, for example, a wrist which is suspected of being fractured, and generate a scan data file comprising the scanned image. The scanner 2202 is operable to provide the scan data file to the cast model generator 2204. The scan data file may comprise, for example, a three-dimensional mapping of the scanned area.

The cast model generator 2204 further comprises a fracture detection module 2206, an immobilization point locator module 2208, a contact area generation module 2210, and a dimension calculation module 2212, the operation of each of which is explained below.

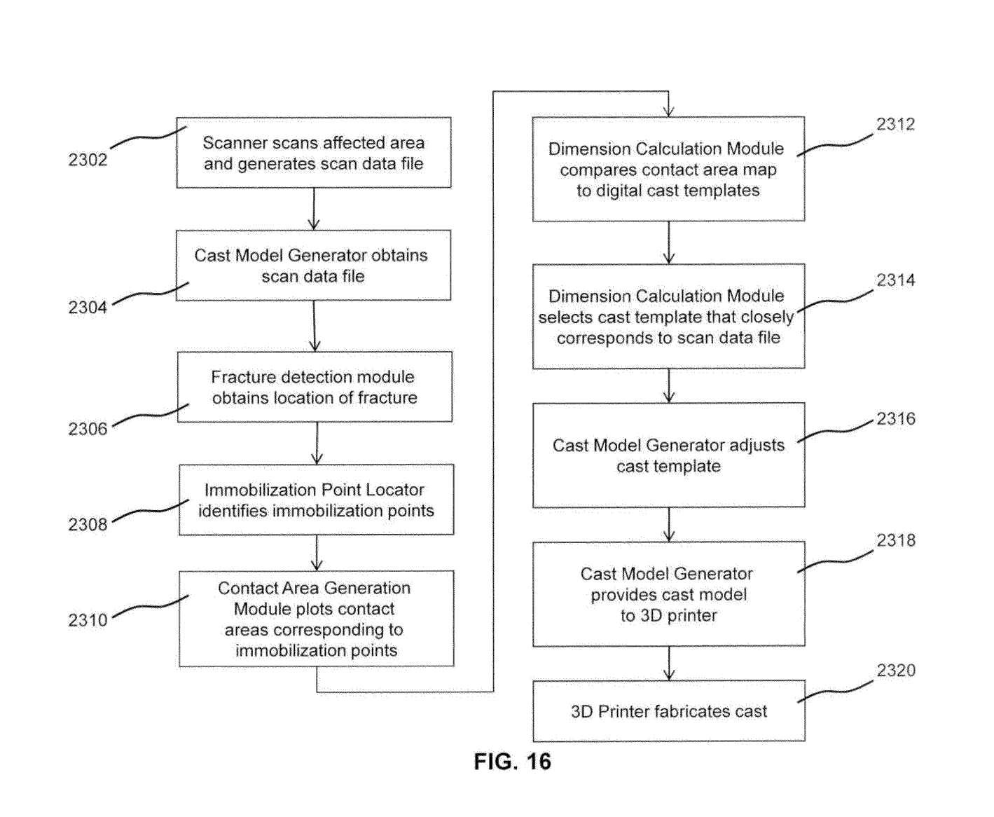

Referring to FIG. 16, example processor executable instructions are provided. At block 2302, the scanner scans the affected area, for example, a potentially fractured wrist. The scanner produces a scan data file of the affected area. At block 2304, the cast model generator 2204 obtains the scan data file and in 2306, the fracture identification module 2206 determines the location of the fracture, or receives an indication of the location of the fracture from a user via the user interface 2216. The fracture identification module 2206 is operable to map the location of a fracture into the scan data file. In one example, the fracture identification module 2206 automatically detects a fracture using, for example, image analysis of an x-ray image to identify an area of lower intensity. Alternatively, or in combination, the fracture detection module 2206 may obtain an input from a user indicating the location of the fracture via the user interface 2216. The fracture detection module 2206 then associates the location of the fracture with the scan data file.

At block 2308, the immobilization point locator 2206 assigns predetermined immobilization points to the scan data file. The immobilization point locator module 2206 may assign predetermined immobilization points obtained from an immobilization point database 2215 to the scan data file based on the morphology of the scanned object and the location of the fracture. For example, if the scanned object is a fractured wrist, the immobilization point locator module 2206 is operable to select predetermined immobilization points corresponding for a wrist from the immobilization point database 2215. The immobilization point locator module 2208 is operable to locate and size the immobilization points with respect to the three-dimensional mapping of the scan data file. This may be performed using, for example, image analysis to map the locations of immobilization points in the immobilization point database 2215 to the corresponding location on the scan data file. The immobilization point locator module 2208 then associates the immobilization points with the scan data file. Alternatively, or in addition, the immobilization point locator module 2206 may receive an input of desired immobilization point locations from a user via the user interface 2216.

The contact area generation module 2210 is operable to obtain the location and size of each of the immobilization points from the scan data file. At block 2310, the contact area generation module plots contact areas corresponding to immobilization points on the scan data file. The contact area generation module 2210 then generates a mapping of contact areas corresponding to the immobilization points, referred to herein as a contact area map. In the example of a fractured wrist, the contact area generation module 2210 generates a contact area map of the contact areas required to immobilize the wrist, as described above with reference to FIG. 9A through 9D.

At block 2312, the dimension calculation module 2212 is operable to obtain the scan data file and the contact area map and compare these to digital cast templates in the cast template database 2214. The dimension calculation module 2212 is operable to select a cast template having dimensions that most closely corresponds to the scan data file and contact area map at block 2314. The dimension calculation module 2212 may compare a set of predetermined measurements to select the most appropriate cast template. For example, the dimension calculation module 2212 may compare the distance between contact areas in each template to the distance between the contact areas generated at block 2210.

The cast templates stored in the cast template database 2214 may comprise templates of casts of various dimensions, for various injuries, and for various areas of the body. For example, the cast template database 2214 may comprise digital cast templates for various sizes of fractured wrists.

The templates may comprise predetermined regions that are adjusted based on the location of the contact areas. For example, a digital cast template for a wrist cast may be adjustable portions wherein the contact areas may be located. At block 2316, the cast model generator 2204 may use a contact area map to modify the adjustable portions of the template such that the contact areas are located to engage with the immobilization points on the cast wearer. The adjusted template is then referred to as a modified cast model. In another example, the cast model generator 2204 may modify regions of the cast template in areas outside of the contact areas.

Alternatively, the digital cast template may be modified by a user via the user interface 2216 to adjust a cast template such that the cast template includes contact areas that engage with immobilization points on the wrist of a wearer.

At block 2318, the cast model generator 2204 is operable to output the modified cast model to the three dimensional printer 2218, or other programmable fabrication tool, to generate a physical cast corresponding to the cast model. At block 2320, the three dimensional printer fabricates the cast according to the modified cast model.

The immobilization point locator module 2208 may alternatively obtain an input from a user via the user interface 2216 indicating the location and size of the immobilization points on the scan data file. The immobilization point locator module 2206 then associates the location of the fracture with the scan data file.

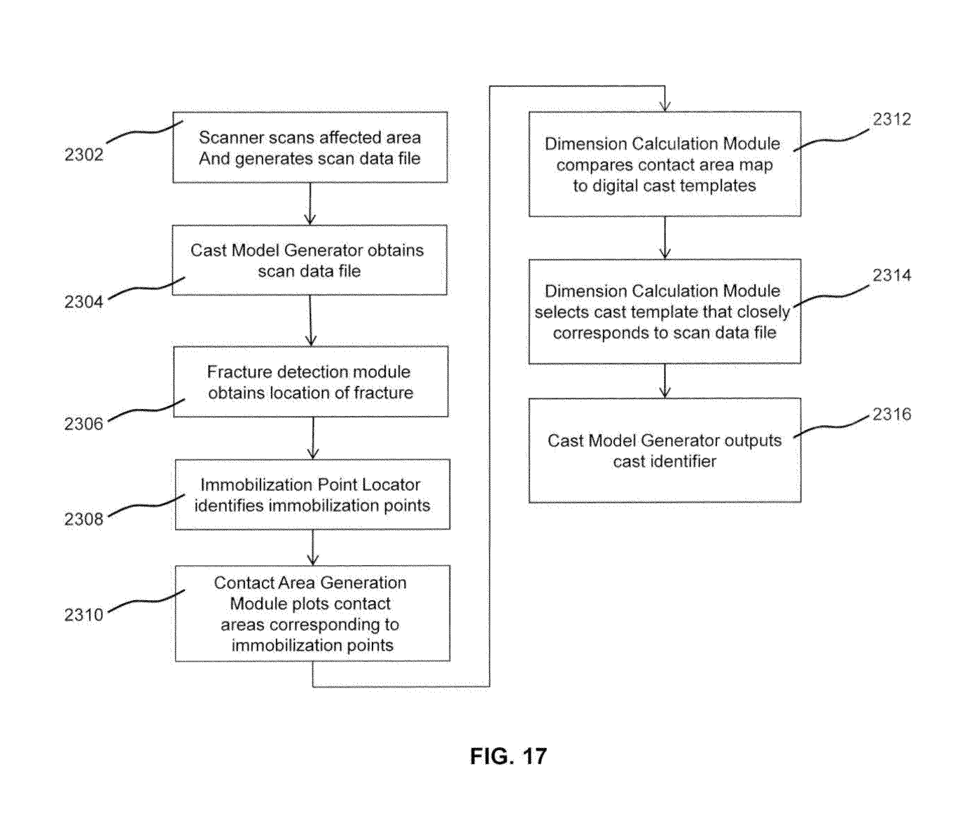

Referring now to FIG. 17, in an alternative example embodiment, after the dimension calculation module 2212 selects a digital cast template at block 2316, the cast model generator 2204 may output, via a display on the user interface 2216, a cast identifier of a pre-manufactured cast corresponding to the cast digital cast template. In this example, the digital cast template comprises predetermined contact areas. As such, a user may, based on the cast identifier outputted by the cast model generator 2204, obtain the selected pre-manufactured cast from a physical cast storage facility.

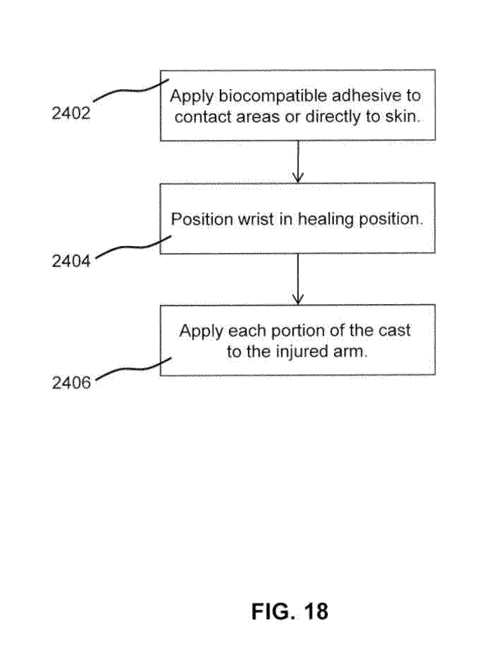

Referring now to FIG. 18, method of physically applying a cast to a wrist using adhesive is provided. At block 2402, a medical adhesive is applied to the contact areas or directly on the skin of the cast wearer. For example, the contact areas or the wearer's arm, or both, may be sprayed with an adhesive. At block 2404, the wrist is positioned in a healing position. At block 2406, each of the inside wrist portion 110 and outside wrist portion 104 are applied to the wearer's wrist. An appropriate adhesive remover may later be applied to the cast 100 and the wearer's arm to remove or disable the adhesive, and allowing the wearer to remove the cast.

Although examples are provided in the context of a cast, it will be appreciated that the principles described herein may be applied to a brace, a splint, or other body member immobilization devices.

It will also be appreciated that although reference is made to immobilizing a wrist, a similar cast comprising a pair of detached opposing members may be formed to immobilize other joints or other portions of a body. For example, an ankle cast may comprise an inside ankle portion and a detached and substantially opposed outside ankle portion that cooperate to substantially immobilize an ankle joint. Similarly, a shoulder cast may comprise two or more portions comprising contact areas to engage the wearer's skin and immobilize a fracture or other injury. A cast may include one or more portions adapted to be independently secured to a wearer.

As such, in a first aspect there is provided an orthopedic cast having a substantially rigid inside wrist portion having a palm support adapted to engage with a palm of a wearer and an inside arm guard adapted to engage with the inside of a forearm of a wearer. The cast further comprises a substantially rigid outside wrist portion having a back hand guard for engaging the back of the wearer's hand and an outside arm guard for engaging the outside of the forearm of the wearer. The inside wrist portion and outside wrist portion being adapted to be secured to the wrist of a wearer to immobilize the wearer's wrist.

In another aspect, the orthopedic cast comprises contact points adapted to engage locations on the wearer's skin at predetermined immobilization points, the contact points being substantially immobile with respect to each other.

In yet another aspect, the orthopedic cast comprises at least one contact point on each of the palm support, inside arm guard, back hand guard, and outside arm guard. In yet another aspect, the areas of the skin facing surface of the cast outside of the contact points are raised so as to be separated from the skin when the cast is being worn.

In yet another aspect, the orthopedic cast is adapted to be secured to the wrist of a wearer by a biocompatible adhesive. The adhesive may be selected from the group consisting of silicone-based adhesives, acrylic-based adhesives, urethane-based adhesives, cyanoacrylate-based adhesives, and epoxies.

In yet another aspect, the inside wrist portion and the outside wrist portion are adapted to engage the wrist of a wearer separately. The orthopedic cast may optionally comprise back hand guard comprises a pair of forwardly-extending hand supports.

In yet another aspect, there is provided a method of selecting an orthopedic cast comprising obtaining a scan data file of a body member, assigning immobilization points to the scan data file of the body member, generating a spatial mapping of contact areas, each contact area being located to engage with one or more of the assigned immobilization points and selecting, from a cast template database, a cast template having contact areas that most closely correspond to the mapping.

In an embodiment, upon selecting the cast template, a cast identifier corresponding to a pre-manufactured cast size is output via the user interface. Upon selecting the cast template, a cast may be generated based on the cast template.

In another embodiment, the cast template is modified such that each contact area on the cast template is brought into closer alignment with the location of its one or more corresponding immobilization points. Upon modifying the cast template, a cast may be generated based on the modified cast template. The cast may be generated using, for example, a three dimensional printer.

In yet another aspect there is provided a system for selecting an orthopedic cast comprising a scanner operable to obtain a scan data file of a body member, and a cast model generator linked to the scanner. The cast model generator comprises an immobilization point locator module operable to assign immobilization points to the scan data file of the body member, a contact area generation module operable to generate a spatial mapping of contact areas, each contact area being located to engage with one or more of the assigned immobilization points, and a dimension calculation module operable to select, from a cast template database, a cast template having contact areas that most closely correspond to the mapping of contact areas.

The dimension calculation module may be further operable to output a cast identifier corresponding to a pre-manufactured cast size via the user interface. The dimension calculation module may further be operable to modify the selected cast template to bring each contact area on the cast template into closer alignment with the location of its one or more corresponding immobilization points.

In yet another aspect, a programmable fabrication unit is in communication with the cast model generator and is operable to generate a cast based on the cast template. In an example embodiment, the programmable fabrication unit is a three dimensional printer.

In yet another aspect, there is provided a kit of parts for an orthopedic cast, comprising a substantially rigid inside wrist portion having a palm support adapted to engage with a palm of a wearer and an inside arm guard adapted to engage with the inside of a forearm of a wearer. The cast further comprises a substantially rigid outside wrist portion having a back hand guard for engaging the back of the wearer's hand and an outside arm guard for engaging the outside of the forearm of the wearer. The inside wrist portion and outside wrist portion being adapted to be secured to the wrist of a wearer to immobilize the wearer's wrist.

In yet another aspect, there is provided a method of applying the cast to a wearer. The method comprises aligning the palm support with the palm of a wearer, aligning the inside arm guard with the inside of the wearer's forearm, aligning the back hand guard with the back of the wearer's hand, aligning the outside arm guard with the forearm of the wearer, and securing each of the inside wrist portion and the outside wrist portion to the arm of the wearer.

What is claimed is:

1. An orthopedic cast comprising:
a substantially rigid inside wrist portion comprising:
a palm support adapted to engage with a palm of a wearer; and
an inside arm guard adapted to engage with the inside of a forearm of a wearer; and
a separate substantially rigid outside wrist portion comprising:
a back hand guard for engaging the back of the wearer's hand; and
an outside arm guard for engaging the outside of the forearm of the wearer,
the inside wrist portion and outside wrist portion being securable to the wrist of a wearer to immobilize the wearer's wrist.

2. The orthopedic cast of claim 1 further comprising contact points adapted to engage locations on the wearer's skin at predetermined immobilization points, the contact points being substantially immobile with respect to each other.

3. The orthopedic cast of claim 2 wherein the cast comprises at least one contact point on each of the palm support, inside arm guard, back hand guard, and outside arm guard.

4. The orthopedic cast of claim 2 wherein the areas of the skin facing surface of the cast outside of the contact points are raised so as to be separated from the skin when the cast is being worn.

5. The orthopedic cast of claim 1 wherein the cast is adapted to be secured to the wrist of a wearer by a biocompatible adhesive.

6. The orthopedic cast of claim 5 wherein the adhesive is selected from the group consisting of silicone-based adhesives, acrylic-based adhesives, urethane-based adhesives, cyanoacrylate-based adhesives, and epoxies.

7. The orthopedic cast of claim 1 wherein the inside wrist portion and the outside wrist portion are adapted to engage the wrist of a wearer separately.

8. The orthopedic cast of claim 1 wherein the back hand guard comprises a pair of forwardly-extending hand supports.

9. A method of selecting an orthopedic cast, the method comprising:
a computing device obtaining a scan data file of a body member;
assigning immobilization points to the scan data file of the body member;
generating a spatial mapping of contact areas, each contact area being located to engage with one or more of the assigned immobilization points; and
selecting, from a cast template database, a cast template having contact areas that most closely correspond to the mapping.

10. The method of claim 9, further comprising:
after selecting the cast template, outputting a cast identifier corresponding to a pre-manufactured cast size via the user interface.

11. The method of claim 9, further comprising:
after selecting the cast template, modifying the cast template such that each contact area on the cast template is brought into closer alignment with the location of its one or more corresponding immobilization points.

12. The method of claim 9, further comprising:
after selecting the cast template, generating a cast based on the cast template.

13. The method of claim 11, further comprising:
after modifying the cast template, generating a cast based on the modified cast template.

14. The method of claim 13 wherein the cast is generated using a three dimensional printer.

15. A system for selecting an orthopedic cast, the system comprising:
a scanner operable to obtain a scan data file of a body member;
a cast model generator linked to the scanner, comprising:
an immobilization point locator module operable to assign immobilization points to the scan data file of the body member;
a contact area generation module operable to generate a spatial mapping of contact areas, each contact area being located to engage with one or more of the assigned immobilization points; and
a dimension calculation module operable to select, from a cast template database, a cast template having contact areas that most closely correspond to the mapping of contact areas.

16. The system of claim 15 wherein the cast model generator is operable to modifying the selected cast template to bring each contact area on the cast template into closer alignment with the location of its one or more corresponding immobilization points.

17. The system of claim 15, further comprising a programmable fabrication unit operable to generate a cast based on the cast template.

18. The system of claim 17 wherein the programmable fabrication unit is a three dimensional printer.

19. A kit of parts for an orthopedic cast, comprising:
a substantially rigid inside wrist portion comprising:
a palm support adapted to engage with a palm of a wearer; and
an inside arm guard adapted to engage with the inside of a forearm of a wearer; and
a separate substantially rigid outside wrist portion comprising:
a back hand guard for engaging the back of the wearer's hand; and
an outside arm guard for engaging the outside of the forearm of the wearer,
the inside wrist portion and outside wrist portion being securable to the wrist of a wearer to immobilize the wearer's wrist.

20. A method of applying the cast of claim 1 to a wearer, the method comprising:
aligning the palm support with the palm of a wearer;
aligning the inside arm guard with the inside of the wearer's forearm;
aligning the back hand guard with the back of the wearer's hand;
aligning the outside arm guard with the forearm of the wearer; and
securing each of the inside wrist portion and the outside wrist portion to the arm of the wearer.
